(19) United States
(12) Plant Patent Application Publication
(10) Pub. No.: US 2005/0114959 P1
(43) Pub. Date: May 26, 2005

(54) Seaside alder named 'September Sun''

(50) ALNUS MARITIMA; SEASIDE ALDER

(76) Inventors: William R. Graves, Ames, IA (US); James A. Schrader, Boone, IA (US)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 10/719,164

(22) Filed: Nov. 22, 2003

(51) Int. Cl. A01H 5/00

(52) U.S. Cl. PLT/226

(57) ABSTRACT

A novel variety of Seaside Alder, Alnus maritima has been identified and asexually propagated. The September Sun Seaside Alder is a large shrub or small tree, that is characterized by being very fast growing, and densely foliated, with symmetrically shaped individual plants.

### BACKGROUND OF THE INVENTION

Seaside alder, specifically Alnus maritima (Marsh. Muhl. ex Nutt) is a relatively rare species in the North American environment that occurs as three disjunct subspecies separated one from another by a large geographic distances. In its native environment the plant appears as a thicket forming large shrub or as a small tree. The seaside alder occurs locally in wet soil or granite outcropping. The seaside alder is principally found locally in Johnston and Byron Counties of Oklahoma, in a region of northwest Georgia, and also locally in Southern Delaware and the Eastern shore of Maryland. It is a shrub that grows very well in water and along the edge of water bodies. Unlike other North American alders that form flowers in the spring, the seaside alder has a late bloom in later summer and early fall. The seaside alder also has characteristic dark green leaves that distinguish it from other alder species.

There has been some successful propagation of the seaside alder by cuttings and seed. The plants taken from each of the three native locations of the plant show slight differences in growth rate and form. The seedlings and cuttings are currently grown in nurseries throughout the Midwest. Nursery grown seaside alder plants will generally grow in most temperate landscapes. Although the native stands of seaside alder seem to occur only in very wet locations, when planted as part of ornamental landscaping, the plants also do very well in cooler and dryer locations.

### DRAWING FIGURES

FIG. 1 is an illustration of the habit of the typical seaside alder September Sun,

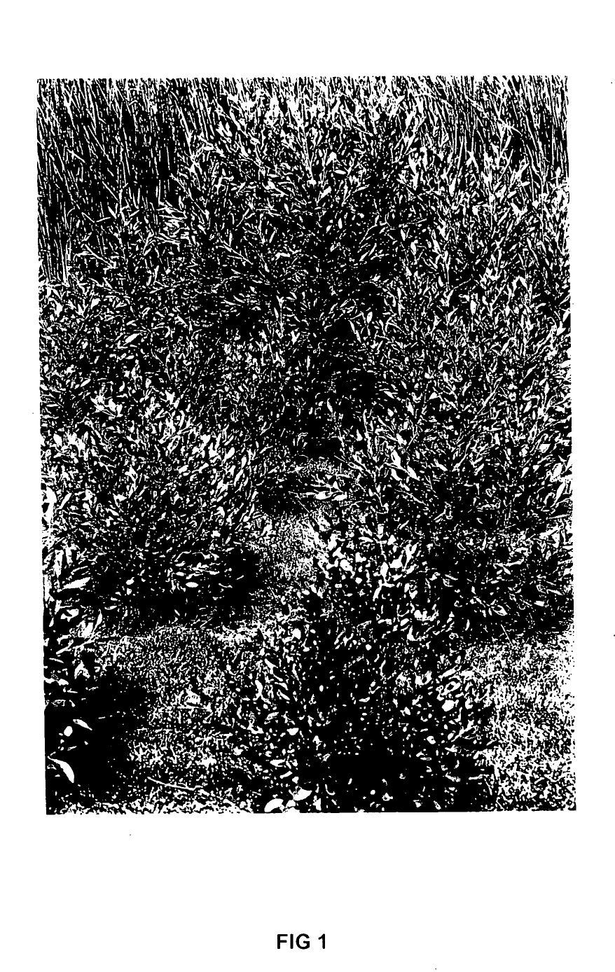

FIG. 2 is an illustration of the fall foliage of the seaside alder September Sun.

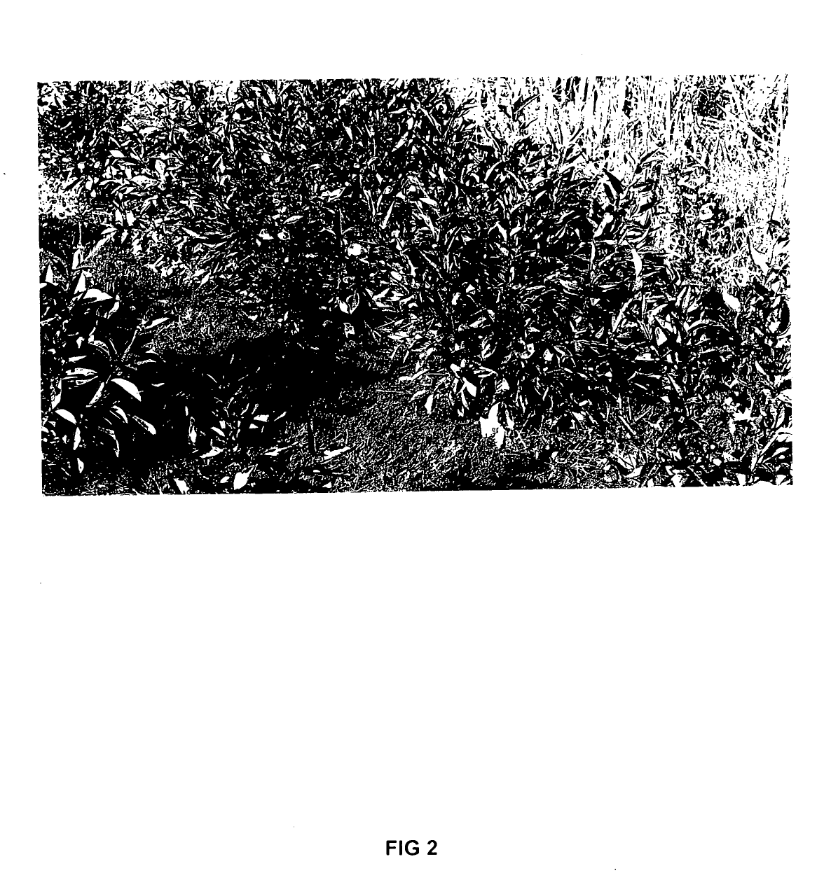

FIG. 3 is an enlarged view of the foliage and catkin of the seaside alder September Sun.

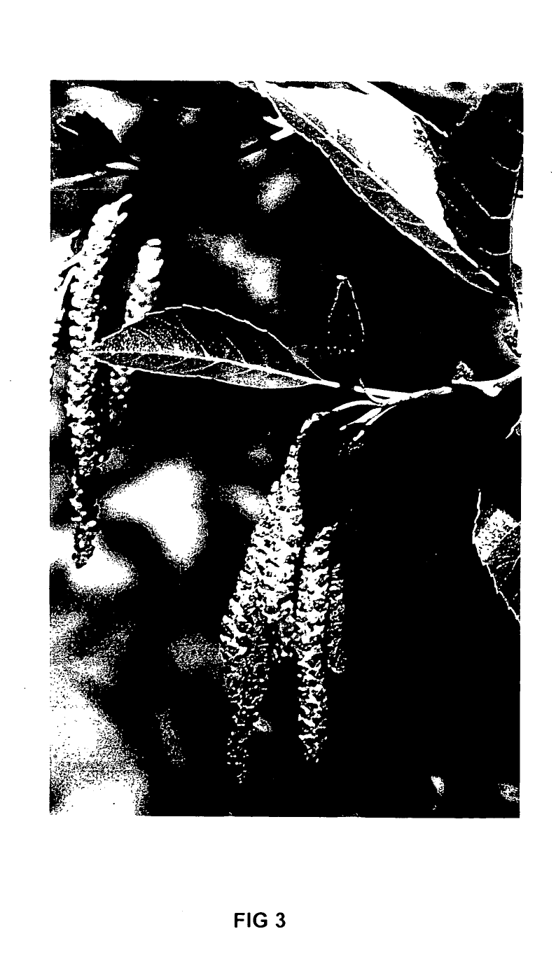

### DESCRIPTION OF THE INVENTION

The present invention relates to a new and distinct variety of seaside alder, Alnus maritima subsp. oklahomensis. The seaside alder named here “September Sun” is characterized by rapid growth, particularly as compared to other plants of its species and subspecies and the development of more marketable ornamental strains as compared to other plants.

September Sun was selected from a trial of seaside alder plants as seedlings of A. maritima subsp. oklahomensis that were grown out on the campus of the Iowa State University at Ames, Iowa. Seeds were collected from a variety of open pollinated A. maritima shrubs and plants growing along the banks of the Blue River near Tishomingo, Okla. Some of the seeds were cultivated into plants that were then scored and evaluated for selection for asexual propagation. September Sun has been asexually propagated with high rates of success by softwood cuttings using the methods described by Schrader and Graves HortScience 35:293-295 (2000). Ramets from the cuttings grow rapidly and can be two meters tall within two years.

In general mature plants of September Sun are large shrubs or small trees with multiple trunks that form broadly rounded upright canopies. Typically the plants grow to a size of seven meters in heights and five meters in width when not crowded by companion plants. As a genotype of A. maritima subsp. oklahomensis trunks of “September Sun” support more leaves and axillary shoots than are found on plants of the other subspecies. This characteristic, when plants of this variety are planted close to each other, can lead to a dense canopy of glossy leaves that are darker in green than the leaves of all other North American alders. Unlike many other A. maritima plants which have been observed, leaves of “September Sun” become mottled blends of yellow, orange, and rich brown under a autumnal conditions in USDA Hardiness Zone 5a. A. maritime, as a species, is monoecious and is the only species of alder native to North America that blooms late in the growing season. Yellow pendulous catkins expand to eight centimeters in length and display staminate flowers from mid-August to late September. The catkins occurs in clusters of two to six on tips of most branches, providing color in the landscape after flowering has ceased on most other trees and shrubs, and before leaf coloration begins during the autumn. Pistillate inflorescences are three to five millimeters in diameter and pink in color. The pistillate inflorescences occur on peduncles that arise from nodes immediately basipetil to the staminate inflorescences. The infructescences of September Sun are medium to dark brown, cone-like strobile. Each of the infructescenses is sixteen to twenty-two millimeters in length and eleven to fourteen millimeters in diameter. The strobili mature one year after pollination and persist on the branches through at least one more season, providing subtle ornamentation on the plant for appeal throughout the year.

The cultivar September Sun differs from other genotypes of its species as it is the fastest growing, most densely foliated, and most symmetrically shaped individual plants that have been observed in field trials including over one thousand plants of the subspecies. During a trial that was conducted over three growing seasons at a site in Ames, Iowa, September Sun grew larger and developed a more symmetrically canopy shape that did other seedlings of A. maritima subsp. oklahomensis, including half-siblings of the original September Sun plant. Shown below in Table 1 is an illustration of the trunk and size characteristics of four representative plants of this subspecies grown in this trial.

TABLE 1

| | Trunk diam^{z} | Canopy height^{y} | |
|---|---|---|---|
| Genotype | (mm) | (cm) | Canopy volume^{x} (m³) |
| “September Sun” | 30.8 a^{w} | 238.9 a | 6.17 a |
| Blue River #6 | 23.0 ab | 166.3 b | 2.35 b |
| Pennington #5 | 24.0 ab | 155.1 b | 1.72 bc |
| Pennington #6 | 19.9 b | 142.1 b | 1.16 c |

^{z}Diameter of the largest trunk at 10 cm above the soil surface.
^{y}Distance from the soil surface to the apex of the tallest shoot.
^{x}Canopy volume was calculated by multiplying the shoot height by the horizontal canopy area (area of an ellipse calculated from the north-south and east-west canopy diameter measurements).
^{w}Means within each column followed by the same letter are not significantly different at P ≦ 0.05 according Student's T-test. N = 1 for “September Sun”, N = 8 for Blue River #6 and Pennington #5, N = 10 for Pennington #6. Dunnett's test for comparing treatment groups against a control (Stevens, 1990) was used to confirm differences between “September Sun” and the three half-sibling groups.

To facilitate identification of the variety, the Macbeth-Munsell Disk Colorimeter was used to specifically identify colors of the important plant parts. The top side of the young leaf emerging from twigs is 5 GY 4/6. The lower side of the young leaf emerging from twigs is 5 GY 5/4. The top side of the mature leaf is 7.5 GY 2/4. The fall foliage is variegated, but the most predominant color is 7.5 Y 7/6. The Male inflorescence or flower cluster is 2.5 Y 7/6. The female inflorescence is 10 RP 4/12. The fully mature fruiting structure, or strobili, is 10 YR 2/1.

1. A new and distinct variety of seaside alder named September Sun, substantially as herein shown and described.
